(19) United States
(12) Patent Application Publication
(10) Pub. No.: US 2006/0093559 A1
(43) Pub. Date: May 4, 2006

(54) Chewing gum composition with vegetal additives

(76) Inventor: Bernd Fabry, Korschenbroich (DE)

Correspondence Address: COGNIS CORPORATION;PATENT DEPARTMENT, 300 BROOKSIDE AVENUE, AMBLER, PA, 19002, US

(21) Appl. No.: 10/541,529

(22) PCT Filed: Dec. 19, 2003
(86) PCT No.: PCT/EP03/14600

§ 371 Date: Jul. 7, 2005

(30) Foreign Application Priority Data
Jan. 8, 2003 (DE) 10300187.5

(51) Int. Cl. A61K 9/68; A61K 36/45; A61K 36/16; A61K 36/48; A61K 36/87; A61K 36/63; A23G 3/00; A61K 36/82

(52) U.S. Cl. 424/48; 424/729; 424/732; 424/757; 424/766; 424/769; 424/752; 426/660

(57) ABSTRACT

A new chewing gum composition is disclosed. The chewing gum formulation contains extracts of plants and/or active principles contained in plant extracts. The plant extracts or plant-derived active principles can be encapsulated. A method for preparing the microcapsules is disclosed.

### FIELD OF THE INVENTION

This invention relates generally to the field of foods and, more particularly, to new chewing gum compositions containing special plant-derived active principles and to the use of these active principles for the production of chewing gum preparations.

### PRIOR ART

Even the natural medicine of many countries still continues to make use of the variety of chemical active principles which are found in plants and which can be obtained from them in more or less pure or concentrated form by extraction with water. Even today, hardly a day goes by without the discovery of a new plant-derived active principle which is subsequently found to show pharmacological activity. Typical examples are the polyphenols found in wine, which prevent cardiac infarct, and vegetable hormones of the isoflavone type which are constituents of purple clover and which are said to be active against a range of diseases from menopausal problems through heart/circulatory diseases to Alzheimer's disease and cancer. If these active principles or the plant extracts containing them have to be orally administered rather than topically applied, so that they have to be metabolized through the metabolism, the galenic commercial forms used are generally sugar-coated pills, tablets and the like. However, such galenic forms often meet with little interest from the consumer because they always carry the taint of a medicament which nobody really wants to take if they do not feel ill.

Accordingly, the problem addressed by the present invention was to provide a new galenic form for the oral application of plant extracts or their active principles which, on the one hand, would be accepted by the consumer and which, on the other hand, would be such as to ensure that the active principles could be readily incorporated and then readily released, preferably with a time delay.

### DESCRIPTION OF THE INVENTION

The present invention relates to new chewing gum compositions containing
(a) a water-insoluble base component,
(b) a water-soluble component and
(c1) extracts of plants selected from the group consisting of Ginkgo biloba, Camellia sinensis, Vaccinium myrtillus, Vinis vitifera, Olea europensis, Trifolium pratense, Salix (alba), Harpagophytum procumbens and mixtures thereof or
(c2) the active principles contained in these plants selected from the group consisting of catechols, flavonoids, quercitrins, resveratrols, flavonoid glycosides, isoflavones, isoflavone glycosides, iridoid glycosides, harpagosides, harpagides, proambides, anthocyano-sides and salicylates and mixtures thereof.

The incorporation of the plant extracts mentioned or the active principles present in them in commercial chewing gum preparations affords a simple possibility acceptable to the consumer of taking plant-derived active principles in dosed form and thus avoiding signs of ageing of the body, especially looseness of the skin, and pursuing a supporting prophylaxis against serious diseases such as, for example, acne vulgaris, arthritis, menopausal problems, heart/circulatory diseases up to Alzheimer's, diabetes and even cancer. The chewing gum compositions may be marketed under the attribute of “health food”, although the active principles may equally well be present as a constituent of traditional chewing gums and may thus make, so to speak, an “incidental” contribution to health care.

Water-Insoluble Base

The water-insoluble base, which is also known as the “gum base” (component a), typically comprises natural or synthetic elastomers, resins, fats and oils, plasticizers, fillers, softeners, dyes and optionally waxes. The base normally makes up 5 to 95% by weight, preferably 10 to 50% by weight and more particularly 20 to 35% by weight of the composition as a whole. In one typical embodiment of the invention, the base consists of 20 to 60% by weight synthetic elastomers, 0 to 30% by weight natural elastomers, 5 to 55% by weight plasticizers, 4 to 35% by weight fillers, 5 to 35% by weight softeners and small amounts of additives, such as dyes, antioxidants and the like, with the proviso that they are soluble in water at best in small quantities.
Elastomers
Suitable synthetic elastomers are, for example, polyisobutylenes with average molecular weights (as measured by GPC) of 10,000 to 100,000 and preferably 50,000 to 80,000, isobutylene/isoprene copolymers (“butyl elastomers”), styrene/butadiene copolymers (styrene:butadiene ratio, for example, 1:3 to 3:1). polyvinyl acetates with average molecular weights (as measured by GPC) of 2,000 to 90,000 and preferably 10,000 to 65,000, polyisoprenes, poly-ethylenes, vinyl acetate/vinyl laurate copolymers and mixtures thereof. Examples of suitable natural elastomers are rubbers, such as for example smoked or liquid latex or guayuls, and natural gums, such as jelutong, lechi caspi, perillo, sorva, massaranduba balata, massaranduba chocolate, nispero, rosindinba, chicle, gutta hang kang and mixtures thereof. The choice of the synthetic and natural elastomers and their mixing ratios essentially depends on whether or not bubbles are to be produced with the chewing gums (bubble gums). Elastomer mixtures containing jelutong, chicle, sorva and massaranduba are preferably used.
Plasticizers
In most cases, the elastomers are too hard or lack plasticity for satisfactory processing, so that it has been found to be of advantage to use special plasticizers which, of course, must also satisfy in particular all requirements relating to acceptability as food additives. In this respect, suitable plasticizers are, above all, esters of resin acids, for example esters of lower aliphatic alcohols or polyols with completely or partly hydrogenated, monomeric or oligomeric resin acids. In particular, the methyl, glycerol or pentaerythritol esters or mixtures thereof are used for this purpose. Alternatively, terpene resins, which may be derived from α-pinene, β-pinene, δ-limonene or mixtures thereof, could also be used.
Fillers and Texturizers
Suitable fillers or texturizers are magnesium or calcium carbonate, ground pumice stone, silicates, especially magnesium or aluminium silicates, clays, aluminium oxides, talcum, titanium dioxide, mono-, di- and tricalcium phosphate and cellulose polymers.
Softeners and Emulsifiers
Suitable softeners or emulsifiers are tallow, hydrogenated tallow, hydrogenated or partly hydrogenated vegetable oils, cocoa butter, partial glycerides, lecithin, triacetin and saturated or unsaturated fatty acids containing 6 to 22 and preferably 12 to 18 carbon atoms and mixtures thereof.
Dyes and Whiteners
Suitable dyes and whiteners are, for example, the FD&C types, plant and fruit extracts permitted for coloring foods and titanium dioxide.

The gum bases may contain waxes or may be wax-free. Examples of wax-free compositions can be found inter alia in U.S. Pat. No. 5,286,500, to the disclosure of which reference is hereby specifically made.

Water-Soluble Component

In addition to the water-insoluble gum base, chewing gum preparations regularly contain a water-soluble component (component b) which is formed, for example, by softeners, sweeteners, fillers, flavors, flavor enhancers, emulsifiers, dyes, acidifiers, antioxidants and the like, with the proviso that the constituents have at least adequate solubility in water. Accordingly, individual constituents may belong both to the water-insoluble phase and to the water-soluble phase, depending on the water solubility of the special representatives. However, combinations may also be used, for example a combination of a water-soluble and a water-insoluble emulsifier, in which case the individual representatives are present in different phases. The water-insoluble component usually makes up 5 to 95% by weight and preferably 20 to 80% by weight of the preparation.
Softeners and Plasticizers
Water-soluble softeners or plasticizers are added to the chewing gum compositions to improve chewability and the chewing feel and are present in the mixtures in quantities of typically 0.5 to 15% by weight. Typical examples are glycerol, lecithin and aqueous solutions of sorbitol, hydrogenated starch hydrolyzates or corn sirup.
Sweeteners
Suitable sweeteners are both sugar-containing and sugar-free compounds which are used in quantities of 5 to 95% by weight, preferably in quantities of 20 to 80% by weight and more particularly in quantities of 30 to 60% by weight, based on the chewing gum composition. Typical saccharide sweeteners are sucrose, dextrose, maltose, dextrin, dried invert sugar, fructose, levulose, galactose, corn sirup and mixtures thereof. Suitable sugar substitutes are sorbitol, mannitol, xylitol, hydrogenated starch hydrolyzates, maltitol and mixtures thereof. Other suitable additives are so-called high-intensity artificial sweeteners (HIAS) such as, for example, sucralose, aspartame, acesulfam salts, alitam, saccharin and saccharin salts, cyclamic acid and salts thereof, glycyrrhicins, dihydrochalcones, thaumatin, monellin and the like either individually or in the form of mixtures. The hydrophobic HIAS, which are the subject of International Patent Application WO 02/091849 A1 (Wrigleys), are also particularly effective. The quantity in which these substances are used is primarily determined by their intensity and is typically in the range form 0.02 to 8% by weight.
Fillers
Fillers are particularly suitable for the production of low-calorie chewing gums and may be selected, for example, from polydextrose, raftilose, raftilin, fructo-oligosaccharides (NutraFlora), palatinose oligosaccharides, guar gum hydrolyzates (Sun Fiber) and dextrins.
Flavors and flavor enhancers
The choice of flavors is virtually unlimited and is not critical to the essence of the invention. They normally make up 0.1 to 15% by weight and preferably 0.2 to 5% by weight of the chewing gum composition. Suitable flavors are, for example, essential oils, synthetic aromas and the like, such as for example peppermint oil, spearmint oil, aniseed oil, Japanese anise oil, caraway oil, eucalyptus oil, fennel oil, citrus oil, wintergreen oil, clove oil, menthol and the like.
Other Auxiliaries and Additives
The chewing gums may additionally contain auxiliaries and additives which are suitable, for example, for dental care, more particularly for controlling plaque and gingivitis, such as for example chlorhexidine, CPC or triclosan. They may also contain pH adjusters (for example buffer or urea), anti-caries agents (for example phosphates or fluorides), biogenic agents (antibodies, enzymes, caffeine, plant extracts), providing these substances are permitted in foods and do not undesirably interact with one another.
   Plant Extracts and Active Principles Present Therein

Plant extracts suitable for the purposes of the invention are plant extracts which contain pharmacological active principles of the polyphenol type, more particularly catechols, flavonoids, quercitrins and resveratrols (for example epicatechol, epigallocatechol, epigallocatechol gallate, theaflavin, theaflavin mono-/digallate), flavonoid glycosides (for example isoquercitrin, kaempferol, kaempferol-3-rhamnoside, isorhamnetin, luteolin, luteolin glycoside, sitosterol glycosides, ginkgolides, bilobalides), anthocyans (for example delphidin), isoflavones and their glycosides (for example daidzein, genestein, formononentin, biochanin A, ononin, sissotrin), iridoid glycosides, harpagosides, harpagides, proambides, anthocyanosides and salicylates (for example salicortin, tremulacin). The following are suitable for this purpose:
Ginkgo biloba
The active ingredients of ginkgo are, above all, flavonoid glycosides (“ginkgoflavonoids”) which include inter alia (iso)quercitin, kaempferol, kaempferol-3-rhamnosides, isorhamnetin, luteolin, luteolin glycosides, sitosterol glycosides and hexacyclic terpene lactones which are known as ginkgolides or bilobalides. The active ingredients of ginkgo are known inter alia for their ability to neutralize free radicals and are used in particular in the prophylaxis or control of signs of ageing of the organism, particularly the skin.
Camellia sinensis

Camellia sinensis is the Latin name for green tea of which the active ingredients (“tea tannins”) are essentially polyphenols, namely epicatechol, epigallocatechol, epigallocatechol gallate, epigallocatechol gallate, theaflavin, theaflavin monogallate A or B and theaflavin digallate which are present in concentrated form in the extracts.

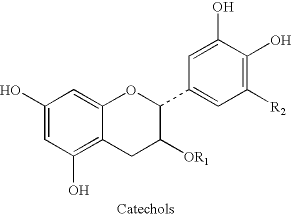

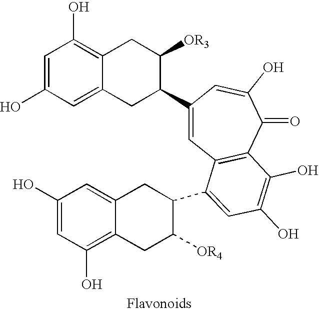

| | R1 | R2 | R3 | R4 |
|---|---|---|---|---|
| (−) Epicatechol | H | H | | |
| (−) Epigallocatechol | H | OH | | |
| (−) Epicatechol gallate | Galloyl | H | | |
| (−) Epigallocatechol gallate | Galloyl | OH | | |
| Theaflavin | | | H | H |
| Theaflavin monogallate A | | | Galloyl | H |
| Theaflavin monogallate B | | | H | Galloyl |
| Theaflavin digallate | | | Galloyl | Galloyl |

The freshly dried tea leaves generally have catechol contents of 20 to 25% by weight, of which the epigallocatechol gallate alone makes up ca. 50 to 70% by weight. Accordingly, preparations containing this active principle in particularly high concentrations are preferred. The active ingredients of green tea are known inter alia for their ability to neutralize free radicals and are used in particular in the prophylaxis or control of signs of ageing of the organism, particularly the skin.
Vaccinium myrtillus
Vaccinium myrtillus is the Latin name for the common bilberry or blueberry. Vaccinium extracts contain as active principles a mixture of at least 15 different anthocyanosides, such as delphinidin for example: 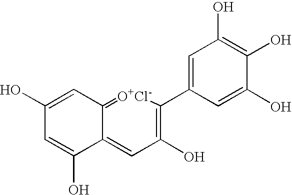
The vaccinium extracts generally contain 20 to 25% by weight of anthocyanosides, 5 to 10% by weight of tannins small quantities of various alkaloids (for example myrtin and epimyrtin), phenolic acids and glycosides with quercitrin, isoquercitrin and hyperoside. The active ingredients of blueberries are also known inter alia for their ability to neutralize free radicals and are used in particular in the prophylaxis or control of signs of ageing of the organism, particularly the skin. Another application is the improvement of sight.
Vinis vitifera
Vinis vitifera is the Latin name for the grapevine. Grape seed extracts contain polyphenols and particularly catechols in quantities of generally up to 15% by weight and preferably in quantities of 10 to 12% by weight; vine leaf extracts contain quercitrins in quantities of normally up to 5% by weight and preferably in quantities of 3 to 4% by weight; and grape skin extracts contain resveratrols in quantities of typically up to 5% by weight and preferably in quantities of 3 to 4% by weight. The individual extracts or active ingredients or (technical) mixtures thereof may be used for the purposes of the invention. The active ingredients of the vine are known inter alia for their ability to neutralize free radicals and may be used in particular in the prophylaxis or control of signs of ageing of the organism, particularly the skin. Another potential application is the prophylaxis of cardiac infarct.
Olea europensis
Olea extracts, which are preferably obtained from the leaves of the olive tree or from the wastewater accumulating in the production of olive oil, are rich in polyphenols and typically contain 1 to 40% by weight, preferably 5 to 30% by weight, more preferably 10 to 25% by weight and most preferably 18 to 22% by weight oleuropein, based on the dry extract. The active principles of the olive tree are known inter alia for their ability to neutralize free radicals and may be used in particular in the prophylaxis or control of signs of ageing of the organism, particularly the skin.
Trifolium pratense

Trifolium pratense is the Latin name for purple clover or common purple trefoil which contains as active principles isoflavones or isoflavone glucosides, above all daidzein, genestein, formononentin, biochanin A, ononin and sissostrin having the following general formula:

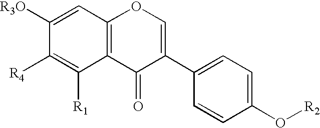

| Isoflavone glucosides | R₁ | R₂ | R₃ | R₄ |
|---|---|---|---|---|
| Daidzein | H | H | Glucose | H |
| Genistin | H | H | Glucose | OH |
| Ononin | H | CH₃ | Glucose | H |
| Sissostrin | H | CH₃ | Glucose | OH |

The active principles of purple clover are used inter alia for the prophylaxis or treatment of menopausal problems, diabetes, Alzheimer's, heart/circulatory disease and cancer.
Salix alba
Salix (pharmacological: Salix cortex) is the Latin name for the plant family of willows which is widespread throughout Europe, Asia and North America. Extracts of willow bark were used for therapeutic purposes in ancient Greece. A willow decoction was widely used in the middle ages as a fever-reducing medicine. In the context of the present invention, salix extracts are understood to be preparations obtained, for example, on the basis of Salix alba, Salix purpurea, Salix fragilis, Salix pentandra and/or Salix daphnoides. The willow extracts may be even replaced by the active principles which are phenolic glycosides and, predominantly, salicylates such as, in particular, salicin (see below), salicortin and tremulacin. 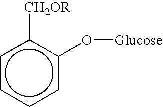
The active principles of the willow are used inter alia for the prophylaxis or treatment of rheumatoid arthritis.
Harpagophytum procumbens
Harpagophytum procumbens (pharmacoligical: Harpagophyti radix) is the Latin name for devil's claw root. At home in the Kalahari Desert and the steppes of Namibia, Madagascar and South Africa, the name devil's claw derives from the hooks with which the fruits are covered. The devil's claw is an established ingredient of traditional African medicine and is prized, above all, for its analgesic and anti-inflammatory properties. However, anti-rheumatic properties have also been attributed to devil's claw, although in vitro and in vivo studies have so far produced contradictory results. In chemical terms, harpagophytum extracts contain above all iridoid glycosides, harpagosides, harpagides and procumbides. 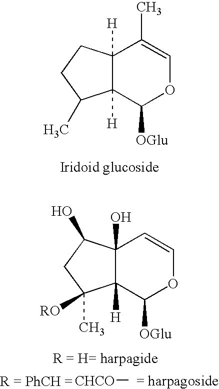
Harpagophytum extracts also contain stachyose, free and glycosylated phytosterols (for example β-sitosterol), flavonoids (for example kaempferol, luteolin), phenolic acids and glycosidic phenylpropanoic acid esters (for example verbacosides, isoacetosides). The active principles in devil's claw are used inter alia for the prophylaxis or treatment of rheumatoid arthritis.
   Extraction

The extracts may be prepared by methods known per se, i.e. for example by aqueous, alcoholic or aqueous/alcoholic extraction of the plants or parts thereof or the leaves or fruit. Suitable extraction processes are any of the usual extraction processes, such as maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux. Percolation is advantageous for industrial use. Fresh plants or parts thereof are suitable as the starting material although dried plants and/or plant parts which may be mechanically size-reduced before extraction are normally used. Any size reduction methods known to the expert, for example freeze grinding, may be used. Preferred solvents for the extraction process are organic solvents, water (preferably hot water with a temperature above 80° C. and more particularly above 95° C.) or mixtures of organic solvents and water, more particularly low molecular weight alcohols with more or less high water contents. Extraction with methanol, ethanol, pentane, hexane, heptane, acetone, propylene glycols, polyethylene glycols, ethyl acetate and mixtures and water-containing mixtures thereof thereof is particularly preferred. The extraction process is generally carried out at 20 to 100° C., preferably at 30 to 90° C. and more particularly at 60 to 80° C. In one preferred embodiment, the extraction process is carried out in an inert gas atmosphere to avoid oxidation of the ingredients of the extract. This is particularly important where extraction is carried out at temperatures above 40° C. The extraction times are selected by the expert in dependence upon the starting material, the extraction process, the extraction temperature and the ratio of solvent to raw material, etc. After the extraction process, the crude extracts obtained may optionally be subjected to other typical steps, such as for example purification, concentration and/or decoloration. If desired, the extracts thus prepared may be subjected, for example, to the selective removal of individual unwanted ingredients. The extraction process may be carried out to any degree, but is usually continued to exhaustion. Typical yields (=extract dry matter, based on the quantity of raw material used) in the extraction of dried leaves are in the range from 3 to 15 and more particularly 6 to 10% by weight. The present invention includes the observation that the extraction conditions and the yields of the final extracts may be selected according to the desired application. These extracts, which generally have active substance contents (=solids contents) of 0.5 to 10% by weight, may be used as such, although the solvent may also be completely removed by drying, more particularly by spray or freeze drying, a deep red colored solid remaining behind. The extracts may also be used as starting materials for producing the pure active substances mentioned above unless they can be synthesized by a more simple and inexpensive method. Accordingly, the active substance content in the extracts may be from 5 to 100% by weight and is preferably from 50 to 95% by weight. The extracts themselves may be present as water-containing preparations and/or as preparations dissolved in organic solvents and as spray-dried or freeze-dried water-free solids. Suitable organic solvents in this connection are, for example, aliphatic alcohols containing 1 to 6 carbon atoms (for example ethanol), ketones (for example acetone), halogenated hydrocarbons (for example chloroform or methylene chloride), lower esters or polyols (for example glycerol or glycols).

Chitosan Microcapsules

In a preferred embodiment of the present invention, the plant extracts or the active principles present in them may be used in encapsulated form, so that they are released with delay during chewing and the taste experience lasts longer. “Microcapsules” are understood by the expert to be spherical aggregates with a diameter of about 0.0001 to about 5 mm which contain at least one solid or liquid core surrounded by at least one continuous membrane. More precisely, they are finely dispersed liquid or solid phases coated with film-forming polymers, in the production of which the polymers are deposited onto the material to be encapsulated after emulsification and coacervation or interfacial polymerization. Chitosan microcapsules and processes for their production are the subject of earlier patent applications filed by applicants [WO 01/01926, WO 01/01927, WO 01/01928, WO 01/01929]. Microcapsules with mean diameters of 0.0001 to 5 mm, preferably 0.001 to 0.5 mm and more particularly 0.005 to 0.1 mm, which consist of a membrane and a matrix containing the active components and which are suitable as component (c), may preferably be obtained by
(a1) preparing a matrix from gel formers, chitosans and active principles,
(a2) optionally dispersing the matrix in an oil phase and
(a3) treating the dispersed matrix with aqueous solutions of anionic polymers and optionally removing the oil phase in the process
   or by
(b1) preparing a matrix from gel formers, anionic polymers and active principles,
(b2) optionally dispersing the matrix in an oil phase and
(b3) treating the dispersed matrix with aqueous chitosan solutions and optionally removing the oil phase in the process
   or by
(c1) processing aqueous active principle preparations with oil components in the presence of emulsifiers to form o/w emulsions,
(c2) treating the emulsions obtained with aqueous solutions of anionic polymers,
(c3) contacting the matrix thus obtained with aqueous chitosan solutions and
(c4) removing the encapsulation products thus obtained from the aqueous phase.
   Gel Formers
   Preferred gel formers for the purposes of the invention are substances which are capable of forming gels in aqueous solution at temperatures above 40° C. Typical examples of such gel formers are heteropolysaccharides and proteins. Preferred thermogelling heteropolysaccharides are agaroses which may be present in the form of the agar agar obtainable from red algae, even together with up to 30% by weight of non-gel-forming agaropectins. The principal constituent of agaroses are linear polysaccharides of D-galactose and 3,6-anhydro-L-galactose with alternate β-1,3- and β-1,4-glycosidic bonds. The heteropolysaccharides preferably have a molecular weight of 110,000 to 160,000 and are both odorless and tasteless. Suitable alternatives are pectins, xanthans (including xanthan gum) and mixtures thereof. Other preferred types are those which—in 1% by weight aqueous solution—still form gels that do not melt below 80° C. and solidify again above 40° C. Examples from the group of thermogelling proteins are the various gelatins.
   Chitosans
   Chitosans are biopolymers which belong to the group of hydrocolloids. Chemically, they are partly deacetylated chitins differing in their molecular weights which contain the following—idealized—monomer unit: 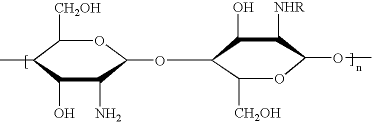
   In contrast to most hydrocolloids, which are negatively charged at biological pH values, chitosans are cationic biopolymers under these conditions. The positively charged chitosans are capable of interacting with oppositely charged surfaces and are therefore used in cosmetic hair-care and body-care products and pharmaceutical preparations. Chitosans are produced from chitin, preferably from the shell residues of crustaceans which are available in large quantities as inexpensive raw materials. In a process described for the first time by Hackmann et al., the chitin is normally first deproteinized by addition of bases, demineralized by addition of mineral acids and, finally, deacetylated by addition of strong bases, the molecular weights being distributed over a broad spectrum. Preferred types are those which have an average molecular weight of 10,000 to 500,000 dalton or 800,000 to 1,200,000 dalton and/or a Brookfield viscosity (1% by weight in glycolic acid) below 5,000 mPas, a degree of deacetylation of 80 to 88% and an ash content of less than 0.3% by weight. In the interests of better solubility in water, the chitosans are generally used in the form of their salts, preferably as glycolates.
   Oil Phase
   Before formation of the membrane, the matrix may optionally be dispersed in an oil phase. Suitable oils for this purpose are, for example, Guerbet alcohols based on fatty alcohols containing 6 to 18 and preferably 8 to 10 carbon atoms, esters of linear C₆₋₂₂ fatty acids with linear C₆₋₂₂ fatty alcohols, esters of branched C₆₋₁₃ carboxylic acids with linear C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of hydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-/di-/triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, Guerbet carbonates, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, ring opening products of epoxidized fatty acid esters with polyols, silicone oils and/or aliphatic or naphthenic hydrocarbons, for example squalane, squalene or dialkyl cyclohexanes.
   Anionic Polymers
   The function of the anionic polymers is to form membranes with the chitosans. Preferred anionic polymers are salts of alginic acid. The alginic acid is a mixture of carboxyl-containing polysaccharides with the following idealized monomer unit: 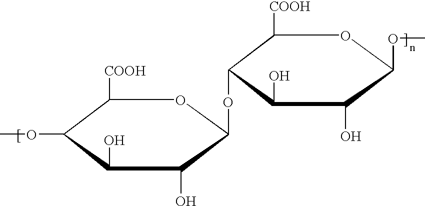
   The average molecular weight of the alginic acid or the alginates is in the range from 150,000 to 250,000. Salts of alginic acid and complete and partial neutralization products thereof are understood in particular to be the alkali metal salts, preferably sodium alginate (“algin”), and the ammonium and alkaline earth metal salts. Mixed alginates, for example sodium/magnesium or sodium/calcium alginates, are particularly preferred. In an alternative embodiment of the invention, however, anionic chitosan derivatives, for example carboxylation and above all succinylation products, are also suitable for this purpose. Alternatively, poly(meth)acrylates with average molecular weights of 5,000 to 50,000 dalton and the various carboxymethyl celluloses may also be used. Instead of the anionic polymers, anionic surfactants or low molecular weight inorganic salts, such as pyrophosphates for example, may also be used for forming the membrane.
   Emulsifiers
   Suitable emulsifiers are, for example, nonionic surfactants from at least one of the following groups:
      products of the addition of 2 to 30 mol ethylene oxide and/or 0 to 5 mol propylene oxide onto linear C₈₋₂₂ fatty alcohols, onto C₁₂₋₂₂ fatty acids, onto alkyl phenols containing 8 to 15 carbon atoms in the alkyl group and onto alkylamines containing 8 to 22 carbon atoms in the alkyl group;
      alkyl and/or alkenyl oligoglycosides containing 8 to 22 carbon atoms in the alk(en)yl group and ethoxylated analogs thereof;
      addition products of 1 to 15 mol ethylene oxide onto castor oil and/or hydrogenated castor oil;
      addition products of 15 to 60 mol ethylene oxide onto castor oil and/or hydrogenated castor oil;
      partial esters of glycerol and/or sorbitan with unsaturated, linear or saturated, branched fatty acids containing 12 to 22 carbon atoms and/or hydroxycarboxylic acids containing 3 to 18 carbon atoms and addition products thereof onto 1 to 30 mol ethylene oxide;
      partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and addition products thereof onto 1 to 30 mol ethylene oxide;
      mixed esters of pentaerythritol, fatty acids, citric acid and fatty alcohol and/or mixed esters of fatty acids containing 6 to 22 carbon atoms, methyl glucose and polyols, preferably glycerol or polyglycerol,
      mono-, di- and trialkyl phosphates and mono-, di- and/or tri-PEG-alkyl phosphates and salts thereof,
      wool wax alcohols,
      polysiloxane/polyalkyl/polyether copolymers and corresponding derivatives,
      block copolymers, for example Polyethyleneglycol-30 Dipolyhydroxystearate;
      polymer emulsifiers, for example Pemulen types (TR-1, TR-2) of Goodrich;
      polyalkylene glycols and
      glycerol carbonate.
         Production of the Chitosan Microcapsules

To produce the microcapsules, a 1 to 10 and preferably 2 to 5% by weight aqueous solution of the gel former, preferably agar agar, is normally prepared and heated under reflux. A second aqueous solution containing the chitosan in quantities of 0.1 to 2 and preferably 0.25 to 0.5% by weight and the active principles in quantities of 0.1 to 25 and preferably 0.25 to 10% by weight is added in the boiling heat, preferably at 80 to 100° C.; this mixture is called the matrix. Accordingly, the charging of the microcapsules with active substances may also comprise 0.1 to 25% by weight, based on the weight of the capsules. If desired, water-insoluble constituents, for example inorganic pigments, may be added at this stage to adjust viscosity, generally in the form of aqueous or aqueous/alcoholic dispersions. In addition, to emulsify or disperse the active substances, it can be useful to add emulsifiers and/or solubilizers to the matrix. After its preparation from gel former, chitosan and active principles, the matrix may optionally be very finely dispersed in an oil phase with intensive shearing in order to produce small particles in the subsequent encapsulation process. It has proved to be particularly advantageous in this regard to heat the matrix to temperatures in the range from 40 to 60° C. while the oil phase is cooled to 10 to 20° C. The actual encapsulation, i.e. formation of the membrane by contacting the chitosan in the matrix with the anionic polymers, takes place in the last, again compulsory step. To this end, it is advisable to treat the matrix optionally dispersed in the oil phase with an aqueous ca. 1 to 50 and preferably 10 to 15% by weight aqueous solution of the anionic polymer at a temperature of 40 to 100° C. and preferably at a temperature of 50 to 600 and, if necessary, to remove the oil phase either at the same time or afterwards. The resulting aqueous preparations generally have a microcapsule content of 1 to 10% by weight. In some cases, it can be of advantage for the solution of the polymers to contain other ingredients, for example emulsifiers or preservatives. After filtration, microcapsules with a mean diameter of preferably about 1 mm are obtained. It is advisable to sieve the capsules to ensure a uniform size distribution. The microcapsules thus obtained may have any shape within production-related limits, but are preferably substantially spherical. Alternatively, the anionic polymers may also be used for the preparation of the matrix and encapsulation may be carried out with the chitosans.

In an alternative process for the production of the microcapsules according to the invention, an o/w emulsion containing an effective quantity of emulsifier besides the oil component, water and the active principles is first prepared. To produce the matrix, a suitable quantity of an aqueous anionic polymer solution is added to this preparation with vigorous stirring. The membrane is formed by adding the chitosan solution. The entire process preferably takes place in the mildly acidic range at pH 3 to 4. If necessary, the pH is adjusted by adding mineral acid. After formation of the membrane, the pH is raised to 5 to 6, for example by adding triethanolamine or another base. This results in an increase in viscosity which can be supported by adding other thickeners such as, for example, polysaccharides, more particularly xanthan gum, guar guar, agar agar, alginates and tyloses, carboxymethyl cellulose and hydroxyethyl cellulose, relatively high molecular weight polyethylene glycol mono- and diesters of fatty acids, polyacrylates, polyacrylamides and the like. Finally, the microcapsules are removed from the aqueous phase by decantation, filtration or centrifuging. The chitosan microcapsules may be present in the chewing gum preparations in quantities of 0.1 to 10% by weight, preferably in quantities of 0.5 to 8% by weight and more particularly in quantities of 1 to 5% by weight.

Production of the Chewing Gum Compositions

The components may be mixed by any known and hence conventional method, including in particular melting to enable components with different melting points to be better processed together. The final preparations may then be converted into individual pieces, for example in the form of strips, blocks, spheres and the like. A basic gum may also be initially produced and then coated with selected components. Typically, the chewing gum preparations have the following composition:
(a) 5 to 49% by weight water-insoluble base component,
(b) 5 to 49% by weight water-soluble component and
(c) 2 to 10% by weight optionally encapsulated plant extracts or their active principles,
   with the proviso that the quantities shown add up to 100% by weight.
   Commercial Applications

The new chewing gum preparations are distinguished by the fact that, by virtue of their content of plant-derived active principles, they counteract signs of ageing of the organism, particularly the skin, and represent a prophylaxis against serious diseases, such as arthritis for example. A preventive effect against cancer has also been suggested by a number of research reports, but has not yet been conclusively demonstrated. Accordingly, the present invention also relates to the use of extracts of plants selected from the group consisting of Ginkgo biloba, Camellia sinensis, Vaccinium myritillus, Vinis vitifera, Olea europensis, Trifolium pratense, Salix (alba), Harpagophytum procumbens and mixtures thereof and the active principles present in them, more particularly those selected from the group consisting of catechols, flavonoids, quercitrins, resveratrols, flavonoid glycosides, isoflavones, isoflavone glycosides, iridoid glycosides, harpagosides, harpagides, proambides, anthocyano-sides and salicylates and mixtures thereof for the production of chewing gum preparations in which they may be present in quantities of 0.1 to 10% by weight, preferably 0.5 to 8% by weight and more particularly 1 to 5% by weight. A number of typical formulations are set out in Table 1 below.

### EXAMPLES

### Example 1

In a 500 ml three-necked flask equipped with a stirrer and reflux condenser, 3 g of agar agar were dissolved in 200 ml of water in boiling heat. First a solution of 10 g of glycerol in 90 ml water and then a preparation of 2.5 g sodium alginate in the form of a 10% by weight aqueous solution, 1 g dried Ginkgo biloba extract, 0.5 g Phenonip® and 0.5 g Polysorbate-20 (Tween® 20, ICI) in 64 g water were added to the mixture over a period of about 30 mins. with vigorous stirring. The matrix obtained was filtered, heated to 60° C. and added dropwise to a 1% by weight solution of chitosan glycolate in water. To obtain microcapsules of the same diameter, the preparations were then sieved.

### Example 2

In a 500 ml three-necked flask equipped with a stirrer and reflux condenser, 3 g of agar agar were dissolved in 200 ml of water in boiling heat. First a solution of 10 g of glycerol in 90 ml water and then a preparation of 2.5 g sodium alginate in the form of a 10% by weight aqueous solution, 1 g dried Camellia sinensis extract, 0.5 g Phenonip® and 0.5 g Polysorbate-20 (Tween® 20, ICI) in 64 g water were added to the mixture over a period of about 30 mins. with vigorous stirring. The matrix obtained was filtered, heated to 60° C. and added dropwise to a 1% by weight solution of chitosan glycolate in water. To obtain microcapsules of the same diameter, the preparations were then sieved.

### Example 3

In a 500 ml three-necked flask equipped with a stirrer and reflux condenser, 3 g of agar agar were dissolved in 200 ml of water in boiling heat. First a solution of 10 g of glycerol in 90 ml water and then a preparation of 2.5 g sodium alginate in the form of a 10% by weight aqueous solution, 1 g dried Vaccinium myrtillus extract, 0.5 g Phenonip® and 0.5 g Polysorbate-20 (Tween® 20, ICI) in 64 g water were added to the mixture over a period of about 30 mins. with vigorous stirring. The matrix obtained was filtered, heated to 60° C. and added dropwise to a 1% by weight solution of chitosan glycolate in water. To obtain microcapsules of the same diameter, the preparations were then sieved.

### Example 4

In a 500 ml three-necked flask equipped with a stirrer and reflux condenser, 3 g of agar agar were dissolved in 200 ml of water in boiling heat. First a solution of 10 g of glycerol in 90 ml water and then a preparation of 2.5 g sodium alginate in the form of a 10% by weight aqueous solution, 1 g dried Vinis vitifera extract, 0.5 g Phenonip® and 0.5 g Polysorbate-20 (Tween® 20, ICI) in 64 g water were added to the mixture over a period of about 30 mins. with vigorous stirring. The matrix obtained was filtered, heated to 60° C. and added dropwise to a 1% by weight solution of chitosan glycolate in water. To obtain microcapsules of the same diameter, the preparations were then sieved.

### Example 5

In a 500 ml three-necked flask equipped with a stirrer and reflux condenser, 3 g of agar agar were dissolved in 200 ml of water in boiling heat. First a solution of 10 g of glycerol in 90 ml water and then a preparation of 2.5 g sodium alginate in the form of a 10% by weight aqueous solution, 1 g dried Trifolium pratense extract, 0.5 g Phenonip® and 0.5 g Polysorbate-20 (Tween® 20, ICI) in 64 g water were added to the mixture over a period of about 30 mins. with vigorous stirring. The matrix obtained was filtered, heated to 60° C. and added dropwise to a 1% by weight solution of chitosan glycolate in water. To obtain microcapsules of the same diameter, the preparations were then sieved.

### Example 6

In a 500 ml three-necked flask equipped with a stirrer and reflux condenser, 3 g of agar agar were dissolved in 200 ml of water in boiling heat. First a solution of 10 g of glycerol in 90 ml water and then a preparation of 2.5 g sodium alginate in the form of a 10% by weight aqueous solution, 1 g dried Olea europensis extract, 0.5 g Phenonip® and 0.5 g Polysorbate-20 (Tween® 20, ICI) in 64 g water were added to the mixture over a period of about 30 mins. with vigorous stirring. The matrix obtained was filtered, heated to 60° C. and added dropwise to a 1% by weight solution of chitosan glycolate in water. To obtain microcapsules of the same diameter, the preparations were then sieved.

A number of Formulation Examples are set out in Table 1 below.

TABLE 1
Formulation Examples (quantities as % by weight)

| Composition | 1 | 2 | 3 | 4 | 5 | 6 | 7 | 8 |
|---|---|---|---|---|---|---|---|---|
| Base | 30.0 | 30.0 | 30.0 | 40.0 | 20.0 | 20.0 | 25.0 | 30.0 |
| Sugar | 51.0 | 51.0 | 51.0 | 42.5 | — | — | — | — |
| Corn sirup | 10.0 | 10.0 | 10.0 | 8.0 | — | — | — | — |
| Sorbitol | — | — | — | — | 51.0 | 51.0 | 47.5 | 44.5 |
| Mannitol | — | — | — | — | 5.0 | 5.0 | 4.3 | 3.6 |
| Glycerol | 1.8 | 1.8 | 1.8 | 1.8 | 8.0 | 8.0 | 8.0 | 7.0 |
| Lycasin/glyc- | — | — | — | — | 8.2 | 8.2 | 8.0 | 7.0 |
| erol mixture | | | | | | | | |
| (1:1) | | | | | | | | |
| Lecithin | — | — | — | — | 0.2 | 0.2 | 0.2 | 0.2 |
| Microcapsules | 1.0 | — | 1.0 | — | 1.0 | — | 1.0 | — |
| of Example 1 | | | | | | | | |
| Microcapsules | — | 1.0 | — | 1.0 | — | 1.0 | — | 1.0 |
| of Example 2 | | | | | | | | |
| Microcapsules | 1.0 | — | — | 1.0 | 1.0 | — | — | 1.0 |
| of Example 3 | | | | | | | | |
| Microcapsules | — | 1.0 | 1.0 | — | — | 1.0 | 1.0 | — |
| of Example 4 | | | | | | | | |
| Microcapsules | 1.0 | — | 1.0 | — | 1.0 | — | 1.0 | — |
| of Example 5 | | | | | | | | |
| Microcapsules | — | 1.0 | — | 1.0 | — | 1.0 | — | 1.0 |
| of Example 6 | | | | | | | | |

| | |
|---|---|
| Water | to 100 |

1-10. (canceled)

11. A chewing gum composition comprising:
(a) a water-insoluble base component;
(b) a water-soluble component; and at least one of (c) and (d), wherein,
(c) comprises an extract of a plant selected from the group consisting of Ginkgo biloba, Camellia sinensis, Vaccinium myrtillus, Vinis vitifera, Olea europensis, Tri-folium pratense, Salix (alba), Harpagophytum procumbens and mixtures thereof and
(d) comprises a plant-derived active principle selected from the group consisting of catechols, flavonoids, quercitrins, resveratrols, flavonoid glycosides, isoflavones, isoflavone glycosides iridoid glycosides, harpagosides, harpagides, proambides, anthocyanosides, salicylates and mixtures thereof.

12. The composition as claimed in claim 11 comprising as component (a) a water-insoluble component selected from the group consisting of natural or synthetic elastomers, resins, fats and oils, plasticizers, fillers, softeners, dyes, waxes and mixtures thereof.

13. The composition as claimed in claim 11 comprising component (a) in a quantity of 5 to 95% by weight.

14. The composition as claimed in claim 11 comprising as component (b) a water-soluble component selected from the group consisting of softeners, sweeteners, fillers, flavors, flavor enhancers, emulsifiers, dyes, acidifying agents, antioxidants and mixtures thereof.

15. The composition as claimed in claim 11 comprising component (b) in a quantity of 5 to 95% by weight.

16. The composition as claimed in claim 11 comprising as component (c) microcapsules with mean diameters of 0.0001 to 5 mm which comprise a membrane and a matrix containing the plant extracts and/or the plant-derived active principles and which are produced by a process comprising a process selected from the group consisting of:
(a)
(a1) preparing a matrix comprising gel formers, chitosans and active principles,
(a2) optionally dispersing the matrix in an oil phase,
(a3) treating the dispersed matrix with an aqueous solution comprising anionic polymers; and
(a4) optionally removing the oil phase,
(b)
(b1) preparing a matrix comprising gel formers, anionic polymers and active principles,
(b2) optionally dispersing the matrix in an oil phase,
(b3) treating the dispersed matrix with an aqueous chitosan solution and d
(b4) optionally removing the oil phase,
(c)
(c1) processing an aqueous active principle preparation with oil components in the presence of emulsifiers to form an o/w emulsion.
(c2) treating the emulsion obtained with an aqukeous solution comprising polymers, to form a matrix,
(c3) contacting the matrix with an aqueous chitosan solution and
(c4) removing the encapsulated product, thus obtained, from the aqueous phase.

17. The composition as claimed in claim 11 which composition contains component (c) in a quantity of 0.1 to 10% by weight.

18. The composition as claimed in claim 11 comprising:
(a) 5 to 49% by weight water-insoluble base component,
(b) 5 to 49% by weight water-soluble component and
(c) 2 to 10% by weight of optionally encapsulated plant extracts and/or their active principles,
with the proviso that the quantities shown add up to 100% by weight.

19. The composition as claimed in claim 12 comprising component (a) in a quantity of 5 to 95% by weight.

20. The composition as claimed in claim 19 comprising as component (b) a water-soluble component selected from the group consisting of softeners, sweeteners, fillers, flavors, flavor enhancers, emulsifiers, dyes, acidifying agents, antioxidants and mixtures thereof.

21. The composition as claimed in claim 20 comprising component (b) in a quantity of 5 to 95% by weight.

22. The composition as claimed in claim 21 comprising as component (c) microcapsules with mean diameters of 0.0001 to 5 mm which comprise a membrane and a matrix containing the plant extracts and/or the plant-derived active principles and which are produced by a process comprising a process selected from the group consisting of:
(a)
(a1) preparing a matrix comprising gel formers, chitosans and active principles,
(a2) optionally dispersing the matrix in an oil phase,
(a3) treating the dispersed matrix with an aqueous solution comprising anionic polymers; and
(a4) optionally removing the oil phase,
(b)
(b1) preparing a matrix comprising gel formers, anionic polymers and active principles,
(b2) optionally dispersing the matrix in an oil phase,
(b3) treating the dispersed matrix with an aqueous chitosan solution, and d
(b4) optionally removing the oil phase,
(c)
(c1) processing an aqueous active principle preparation with oil components in the presence of emulsifiers to form an o/w emulsion.
(c2) treating the emulsion obtained with an aqukeous solution comprising polymers, to form a matrix,
(c3) contacting the matrix with an aqueous chitosan solution and
(c4) removing the encapsulated product, thus obtained, from the aqueous phase.

23. The composition as claimed in claim 22 which composition contains component (c) in a quantity of 0.1 to 10% by weight.

24. The composition as claimed in claim 23 comprising:
(a) 5 to 49% by weight water-insoluble base component,
(b) 5 to 49% by weight water-soluble component and
(c) 2 to 10% by weight of optionally encapsulated plant extracts and/or their active principles,
with the proviso that the quantities shown add up to 100% by weight.

25. The chewing gum composition of claim 11, wherein, the water-insoluble base component comprises from 10% to 50% by weight of the composition.

26. The chewing gum composition of claim 25, wherein, the water-soluble component comprises from 20% to 80% by weight of the composition.

27. The composition of claim 16, wherein, the microcapsules have a mean diameter of from 0.001 mm to 0.5 mm.

28. The composition of claim 27, wherein, the microcapsules have a mean diameter of from 0.005 mm to 0.1 mm.
